(19) United States
(12) Patent Application Publication
(10) Pub. No.: US 2019/0263911 A1
(43) Pub. Date: Aug. 29, 2019

(54) ANTI-BAG3 ANTIBODIES IN COMBINATION WITH INHIBITORS OF IMMUNE CHECK-POINT FOR THERAPEUTIC USE

(71) Applicant: BIOUNIVERSA S.R.L., Montoro AV (IT)

(72) Inventors: Maria Caterina TURCO, Avellino (IT); Vincenzo DE LAURENZI, Pescara (IT); Liberato MARZULLO, Scalea (IT); Alessandra ROSATI, Baronissi (IT)

(21) Appl. No.: 16/345,909

(22) PCT Filed: Nov. 6, 2017
(86) PCT No.: PCT/EP2017/078264

§ 371 Date: Apr. 29, 2019

(30) Foreign Application Priority Data
Nov. 7, 2016 (IT) 102016000111877

(51) Int. Cl. C07K 16/28; A61P 35/00; C07K 16/30; A61K 9/00

(52) U.S. Cl. CPC C07K 16/2818; A61K 9/0053; C07K 16/303; A61P 35/00

(57) ABSTRACT

The present invention relates to a combination comprising anti-BAG3 antibodies and inhibitors of the immune check-point, to pharmaceutical formulation comprising said combination, optionally with an pharmaceutically acceptable excipient and to its use in the treatment of neoplastic diseases.

The present invention relates to a combination comprising anti-BAG3 antibodies and inhibitors of the immune check-point, to pharmaceutical formulation comprising said combination, optionally with an pharmaceutically acceptable excipient and to its use in the treatment of neoplastic diseases.

### BACKGROUND OF THE INVENTION

BAG3 protein is a 74 kDa cytoplasmic protein which belongs to the family of co-chaperons that interact with the ATPase domain of the protein HSP70 (Heat Shock Protein) through the structural domain known as the BAG domain (amino acids 110-124). Furthermore, BAG3 protein contains a WW domain (Trp-Trp), a proline-rich region (PXXP), and two conserved motifs IPV (Ile-Pro-Val), which can mediate the binding to other proteins. Thanks to the nature of BAG3 protein as an adapter, attributable to the presence of many functional domains, such protein can therefore interact with different proteins.

In humans, bag3 gene expression is constitutive for a few kinds of normal cells, including myocytes, while mutations thereof are associated with diseases of the skeletal and cardiac muscles. Furthermore, BAG3 protein is expressed in many types of primary tumours or tumour cell lines (lymphoid or myeloid leukemias, neuroblastoma, pancreatic cancer, thyroid cancer, breast cancer and prostate cancer, melanoma, osteosarcoma, glioblastoma and tumours of the kidney, colon, ovary, etc.) (Rosati A. et al Cell Death Dis. 2011 Apr. 7; 2:e141).

In normal cell types, such as leukocytes, epithelial cells and glial cells and cells of the retina, bag3 gene expression can be induced by stressors, such as oxidants, high temperatures, lack of serum, heavy metals, HIV-1 infections, etc. These findings indicate that bag3 gene expression regulation is an important component in the cellular response to stress and is correlated with the presence of elements that respond to the transcription factor HSF1 (Heat Shock Transcription Factor), which is activated in various forms of cellular stress in bag3 gene promoter. Moreover, due to the presence of many protein-protein interaction domains in the structure thereof, BAG3 protein influences cell survival in different types of cells, interacting with different molecular partners (Rosati A. et al Cell Death Dis. 2011 Apr. 7; 2:e141). The first mechanism reported in relation to BAG3 anti-apoptotic activity was identified in osteosarcoma and melanoma cells, where it was observed that BAG3 protein modulates the activation of transcription factor NF-kB and cell survival (Ammirante M. et al. Proc Natl Acad Sci USA. 2010; 107(16):7497-502.). A different molecular mechanism has been described in glioblastoma cells, where BAG3 protein cooperates in a positive way with HSP70 protein to maintain BAX protein in the cytosol and prevent the translocation thereof into the mitochondria (Festa M. et al. Am J Pathol. 2011; 178(6):2504-12). Finally, in some tumours, BAG3 has been shown to regulate proteins that modulate cell adhesion.

The presence of cytoplasmic BAG3 protein has also been described in many different cellular systems and has been associated, not only with various tumours, but also in pathologies in general related to cell survival. Furthermore, patent application n. WO2011/067377 describes extracellular BAG3 protein, secreted by some cell types, as a biochemical marker in serum, which is highly specific for the diagnosis of certain pathological conditions, such as cardiac pathologies and pancreatic tumour.

It has recently been reported that BAG3 protein is expressed in 346/346 patients with pancreatic ductal adenocarcinoma (PDAC) and is released by the cells of the pancreatic tumour, but such protein is not expressed in either the surrounding non-neoplastic tissues or in a normal pancreas; likewise, it has been reported that the levels of BAG3 expression are related to patient survival. The results of the study demonstrate that the use of specific siRNA molecules for BAG3 mRNA can silence bag3 gene expression and induce cell death, confirming that BAG3 protein is an important survival factor for pancreatic tumour cells and that the down-regulation thereof, when combined with gemcitabine, may contribute to the eradication of the tumour cells (Rosati A. et al. Am J Pathol. 2012 November; 181(5): 1524-9).

Moreover, in a recent paper (Rosati A. et al. Nat Commun. 2015 Nov. 2; 6:8695), we have reported that PDAC-released BAG3 binds macrophages inducing their activation and the secretion of PDAC supporting factors. We have also identified IFITM-2 as a BAG3 receptor and showed that it signals through PI3K and the p38 MAPK pathways. Finally, we have showed that the use of a mouse monoclonal anti-BAG3 antibody results in reduced tumour growth and prevents metastasis formation in three different mouse models. We have therefore identified a paracrine loop involved in PDAC growth and metastatic spreading, and showed that an anti-BAG3 antibody has therapeutic potential (Rosati A. et al. Nat Commun. 2015 Nov. 2; 6:8695). Indeed, we showed that an anti-BAG3 antibody blocked BAG3 activity on macrophages. In vivo, we showed the ability of this antibody to block tumour growth in different animal models, including a model of patient-derived xenograft and a syngeneic model. This last model is of great importance since mice have an intact immune system.

Conventional chemotherapy treatments for tumour pathologies, pose numerous drawbacks linked to side effects and are not, at present, definitive means of treating such pathologies.

In recent years, immune checkpoint inhibitors, that are molecuses that inhibit/block the immune checkpoint system, have emerged as effective therapies for advanced neoplasias; among these are therapeutic antibodies that block cytotoxic T lymphocyte associated antigen 4 (CTLA4) and programmed cell death protein 1 (PD-1), that have been used for several tumours (Topalian S L et al., Nat Rev Cancer. 2016 May; 16(5):275-87.). PD-1 (Programmed cell Death protein, CD279), (a member of the B7/CD28 family of receptors, is a monomeric molecule expressed on the cell surface of activated leucocytes, including T, B, NK and myeloid-derived suppressor cells, whose expression is finely regulated by an interplay between genetic and epigenetic mechanisms. Known ligands of PD-1 are PD-L1 and PD-L2 (Farkona S. et al., BMC Med. 2016 May 5; 14:73).

PD-L1 (Programmed cell Death Protein Ligand 1, B7H1, CD274) is expressed at low levels, and up-regulated upon cell activation, on haematopoietic cells, including T, B, myeloid, and dendritic cells, and non-hematopoietic (such as lung, heart, endothelial, pancreatic islet cells, keratinocytes) and specially cancer cells. PD-L2 (Programmed cell Death Protein Ligand 2, B7-DC, CD273) is expressed on macrophages, dendritic cells (DCs), activated CD4+ and CD8+ lymphocytes and some solid tumours (ovarian carcinoma, small cell lung cancer, oesophageal cancer). PD-L1 and PD-L2 expression has also been detected on normal and cancer-associated fibroblasts Both PD-L1 and PD-L2 interact with additional receptors: PD-L1 with the CD28 ligand CD80 and PD-L2 with Repulsive Guidance Molecule (RGM) b, expressed on macrophages and other cell types. The cytoplasmic tail of PD-1 contains an Immunoreceptor Tyrosine-based Inhibition Motif (ITIM) and an immunoreceptor tyrosine-based switch motif (ITSM). In T lymphocytes, PD-1 interaction with its ligands results in the phosphorylation of two tyrosines at the intracellular tail of PD-1; the recruitment of SH2 domain-containing protein tyrosine phosphatases (SHP-1 and/or SHP-2) to the ITSM cytoplasmic region of PD-1 then inhibits downstream signals of the T-cell receptor, thereby inhibiting T cell proliferation and cytokine production. PD-1 exerts also other effects on T cells: for example, by inhibiting Akt and Ras pathways, PD-1 triggering suppresses transcription of the ubiquitin ligase component SKP2: this results in impairing SKP2-mediated degradation of p27(kip1), an inhibitor of cyclin-dependent kinases, and thereby in blocking cell cycle progression. In addition, PD-1 can promote apoptosis by more than one mechanism Besides directly inhibiting T cell activation, PD-1 triggering by PD-L1 can induce the development of T regulatory cells (Treg), key mediators of peripheral tolerance that actively suppress effector T cells. Treg induction by PD-1 triggering is mediated by modulation of key signalling molecules, such as phospho-Akt, whose levels are kept low by the PD-1-induced activity of PTEN. Several types of cancer cells do express PD-L1. Furthermore, non neoplastic cells (endothelial cells, leucocytes, fibroblasts) in the tumour microenvironment can also express PD-L1. This suggests that they can tolerise tumour-infiltrating PD-1+T lymphocytes (TILs), and/or induce Treg development; indeed a growing body of evidence indicate that treatment of patients affected by some cancer types (melanoma, renal carcinoma, Non-Small Cell Lung Cancer, etc.) with anti-PD-1/PD-L1 monoclonal antibodies (mAbs) can reduce tumour growth.

Currently, more than 100 clinical trials are investigating PD-1 and PD-L1 blocking clinical efficacy in a variety of cancers. However, despite the very encouraging results, it is clear that a) not all tumour types show significant response to anti-PD-1 or anti-PD-L1 mAbs, and b) in the subsets of responding cancers, not all patients are responsive and some responses are very partial. These pieces of evidence, in conjunction with the uncertainty, at this stage of the studies, on the durability of responses, indicate the need for effective therapeutic combinations between anti-PD-1/PD-L1 mAbs and tools that act on other pathways (Topalian S L et al. Cancer Cell. 2015 Apr. 13; 27(4):450-61).

There is therefore an evident need for a new and improved therapeutic treatment which has the advantage of being highly specific and having few or no side effects, as compared with the conventional, commonly known therapies used for the treatment of neoplastic diseases.

### Definitions

Unless otherwise defined, all terms of art, notations and other scientific terminology used herein are intended to have the meanings commonly understood by those persons skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “pharmaceutically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

The term “simultaneous, separate or sequential administration” herein refers to administration of the first and second compound at the same time or in such a manner that the two compounds act in the patient's body at the same time or administration of one compound after the other compound in such a manner to provide a therapeutic effect. In some embodiments the compounds are taken with a meal. In other embodiments, the compounds are taken after a meal, such as 30 minutes or 60 minutes after a meal. In some embodiments, one compound is administered to a patient for a time period followed by administration of the other compound.

The terms “approximately” and “about” herein refer to the range of the experimental error, which may occur in a measurement.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of” and “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms. The term “antibody” as used herein includes “fragments” or “derivatives”, which have at least one antigen binding site of the antibody and/or show the same biological activity.

An antibody preferably comprises at least one heavy immunoglobulin chain and at least one light immunoglobulin chain. An immunoglobulin chain comprises a variable domain and optionally a constant domain. A variable domain may comprise complementarity determining regions (CDRs), e.g. a CDR1, CDR2 and/or CDR3 region, and framework regions.

The term “humanized antibody” refers to an antibody of human origin, whose hypervariable region has been replaced by the homologous region of non-human monoclonal antibodies.

The term “chimeric antibody” refers to an antibody containing portions derived from different antibodies.

The term “recombinant antibody” refers to an antibody obtained using recombinant DNA methods.

The term “scFv fragment” (single chain variable fragment) refers to immunoglobulin fragments only capable of binding with the antigen concerned. ScFv fragments can also be synthesised into dimers (diabodies), trimers (triabodies) and tetramers (tetrabodies) using peptide linkers.

The terms “Fab fragment” (antigen-binding fragment) and “Fab2 fragment” refer to immunoglobulin fragments consisting of a light chain linked to the Fc fragment of the adjacent heavy chain, and such fragments are monovalent antibodies. When the Fab portions are in pairs, the fragment is called Fab2.

The term “hybridoma” refers to a cell producing monoclonal antibodies. The term “monospecific antibodies” refers to antibodies that all have affinity for the same antigen.

The term “multispecific antibodies” refers to antibodies that have affinity for several antigens.

The term “bispecific antibody” refers to an antibody that has affinity for two different antigens.

The term “immune checkpoint inhibitor” refers to a type of drug that blocks certain proteins made by some types of immune system cells, such as T cells, and some cancer cells.

The term “A2A” refers to the Adenosine A2A receptor.

The term “B7-H3, also called CD276” refers to a protein expressed on solid tumours that participate in the regulation of T-cell-mediated immune response.

The term “B7-H4”, also called VTCN1, refers to a molecule expressed by tumour cells and tumour-associated macrophages that plays a role in tumour escape.

The term “BTLA”, also called CD272, refers to a B and T Lymphocyte Attenuator.

The term “CTLA-4”, also called CD152, refers to Cytotoxic T-Lymphocyte-Associated protein 4.

The term “IDO” refers to Indoleamine 2,3-dioxygenase, that is a tryptophan catabolic enzyme with immune-inhibitory properties.

The term “KIR”, Killer-cell Immunoglobulin-like Receptor, refers to a receptor for MHC Class I molecules on Natural Killer cells.

The term “LAG3”, refers to Lymphocyte Activation Gene-3.

The term “TIM-3” refers to T-cell Immunoglobulin domain and Mucin domain 3.

The term “VISTA (protein)”, refers to V-domain Ig suppressor of T cell activation.

The term “sequence identity” between two polypeptide sequences, indicates the percentage of amino acids that are identical between the sequences.

### DESCRIPTION OF THE FIGURES

FIG. 1. Anti-BAG3 mAb induces PD-1 expression in pancreatic cancer allografts in immunocompetent mice.

FIG. 2. Anti-BAG3 mAb induces PD-1 expression in pancreatic cancer allografts in immunocompetent mice.

FIG. 3. Combination of anti-BAG3 and anti-PD-1 arrest tumour growth in pancreatic cancer allografts in immunocompetent mice.

### DISCLOSURE OF THE INVENTION

It has been surprisingly found that a combination comprising specific anti-BAG3 antibodies and an inhibitor of the immune check point, for example anti-PD-1 antibodies, is effective in arresting the tumour growth in pancreatic cancer.

In particular, it has been for the first time observed that anti-BAG3 antibodies, in addition to blocking the interaction between BAG3 protein and its receptor on the macrophage surfaces, are able to induce the production of PD-1 and/or PDL-1 molecules on the tumour cell surface and therefore to enhance the effect of anti-PD-1 or anti-PDL-1 antibodies on tumoural cells.

One hypothesis of mechanism for that can be related to the down modulation of several cytokines in the tumour microenvironment induced by anti-BAG3 antibodies (Rosati A. et al. Nat Commun. 2015 Nov. 2; 6:8695).

The experimental data reported in the present invention therefore demonstrates that the claimed combination is particularly effective in the treatment of neoplastic disease, in particular pancreatic cancer.

Therefore, a first embodiment of the present invention relates to a combination comprising an anti-BAG3 antibody or a fragment thereof and at least one inhibitor of the immune check point.

Anti-BAG3 antibodies or fragments thereof according to the invention may be polyclonal or monoclonal antibodies. Monoclonal antibodies are preferred.

Preferably the anti-BAG3 antibodies or fragments thereof are humanized antibodies which comprises:

a) a heavy chain amino acid sequence as encoded by SEQ ID NO: 12 or at least the variable domain thereof or an amino acid sequence having a sequence identity of at least 80% thereof, and

b) a light chain amino acid sequence as encoded by SEQ ID NO: 20 or at least the variable domain thereof or an amino acid sequence having a sequence identity of at least 80% thereof.

As used herein, sequence identity between two polypeptide sequences, indicates the percentage of amino acids that are identical between the sequences, preferably over the entire length of the amino acid sequences as encoded by SEQ ID NO: 12 and SEQ ID NO: 20.

Preferred polypeptide sequences of the invention have a sequence identity of at least 85%, more preferably 90%, even more preferably 93%, 95%, 96%, 97%, 98% or 99%.

In a preferred embodiment of the present invention said amino acid sequence having a sequence identity of at least 80% with respect to SEQ ID N. 12 is selected from SEQ ID N. 14, SEQ ID N: 16 or SEQ ID N. 18.

In a further preferred embodiment said amino acid sequence having a sequence identity of at least 80% with respect to SEQ ID N. 20 is selected from SEQ ID N. 22, SEQ ID N: 24 or SEQ ID N. 26.

In a preferred embodiment the antibody of the present invention is the antibody wherein the heavy chain amino acid sequence is encoded by SEQ ID NO. 18 and the light chain amino acid sequence is encoded by SEQ ID NO 22 or SEQ ID N. 26.

In a preferred embodiment, the heavy chain amino acid sequence or at least the variable domain thereof or an amino acid sequence having a sequence identity of at least 80% thereof, comprises the CDRs regions having the following amino acid composition: H-CDR1 comprises the amino acids GFNIKDTYMY (SEQ ID N. 3), H-CDR2 comprises the amino acids GVDPANGNTRYDPKFQG (SEQ ID N. 4), H-CDR3 comprises the amino acids DGAMDY (SEQ ID N. 5) and the light chain amino acid sequence or at least the variable domain thereof or an amino acid sequence having a sequence identity of at least 80% thereof, comprises the CDRs regions having the following amino acid composition: L-CDR1 comprises the amino acids KSSQSLLYSSNQKNYLA (SEQ ID N. 6), L-CDR2 comprises the amino acids WASTRES (SEQ ID N. 7) and L-CDR3 comprises the amino acids QQYYTYPLT (SEQ ID N. 8).

A further embodiment of the present invention, is an antibody or a fragment thereof which binds to the BAG3 protein and which comprises:

a) a heavy chain nucleotide sequence as encoded by SEQ ID NO: 11 or at least the variable domain thereof or a nucleotide sequence having a sequence identity of at least 80% thereof, and

b) a light chain nucleotide sequence as encoded by SEQ ID NO: 19 or at least the variable domain thereof or a nucleotide sequence having a sequence identity of at least 80% thereof.

As used herein, “sequence identity” between two nucleotide sequences, indicates the percentage of nucleotides that are identical between the sequences, preferably over the entire length of the nucleotide sequences as encoded by SEQ ID NO: 11 and SEQ ID NO: 19. Preferred nucleotide sequences of the invention have a sequence identity of at least 85%, more preferably 90%, even more preferably 93%, 95%, 96%, 97%, 98% or 99%.

In a preferred embodiment of the present invention said nucleotide sequence having a sequence identity of at least 80% with respect to SEQ ID N. 11 is selected from SEQ ID N. 13, SEQ ID N: 15 or SEQ ID N. 17.

In a further preferred embodiment said amino acid sequence having a sequence identity of at least 80% with respect to SEQ ID N. 19 is selected from SEQ ID N. 21, SEQ ID N: 23 or SEQ ID N. 25.

In a preferred embodiment the antibody of the present invention is the antibody wherein the heavy chain amino acid sequence is encoded by SEQ ID NO. 17 and the light chain amino acid sequence is encoded by SEQ ID NO 21 or SEQ ID N. 25.

Inhibitors of the immune check point according to the present invention may be antibodies or fragments thereof which bind to following immune checkpoint molecules: PD-1, PDL-1, A2AR, B7-H3, B7-H4, BTLA, CTLA-4, IDO, KIR, LAG3, TIM-3 or VISTA protein, preferably PD-1, PDL-1 or CTLA-4 protein.

In a preferred embodiment of the present invention the at least one inhibitor of the immune check point is selected from an antibody, a protein, a small molecule and/or a si-RNA.

More preferably, said at least one inhibitor of the immune check point is an antibody.

Preferably said antibodies are monoclonal antibodies selected from Pidilizumab, Atezolizumab, Avelumab, Durvalumab, Ipilimumab, Tremelimumab, Nivolumab and Pembrolizumab.

In a preferred embodiment the combination of the present invention comprises one anti-BAG3 antibody or a fragment thereof and one anti-PD-1 antibody or a fragment thereof.

Preferably said anti-PD1 antibody is selected from Nivolumab and Pembrolizumab, more preferably Nivolumab.

In a further preferred embodiment the aforesaid combination comprises one anti-BAG3 antibodies or a fragment thereof and one anti-PDL-1 antibody and/or a fragment thereof.

Preferably said anti-PDL-1 antibody is selected from Atezolizumab, Avelumab and Durvalumab, more preferably Atezolizumab.

For the purpose of the present invention the antibodies are preferably selected from the group consisting of murine antibody, recombinant antibody, humanized or fully human antibody, chimeric antibody, multispecific antibody, in particular bispecific antibody, or a fragment thereof.

Monoclonal antibodies may be produced by any suitable method such as that of Köhler and Milstein (1975) or by recombinant DNA methods. Monoclonal antibodies may also be isolated from phage antibody libraries using techniques described in Clackson et al. (1991).

Humanized forms of the antibodies may be generated according to the methods known in the art, (Kettleborough C. A. et al., 1991), such as chimerization or CDR grafting. Alternative methods for the production of humanized antibodies are well known in the art and are described in, e.g., EP 0239400 and WO 90/07861. Human antibodies can also be derived by in vitro methods. Suitable examples include but are not limited to phage display, yeast display, and the like.

According the present invention “chimeric antibody” relates to antibodies comprising polypeptides from different species, such as, for example, mouse and human. The production of chimeric antibodies is described, for example, in WO 89/09622.

The term antibody includes “fragments” or “derivatives”, which have at least one antigen binding site of the antibody.

According to a preferred embodiment the antibody or fragment thereof may be a Fab fragment, a Fab′ fragment, a F(ab′) fragment, a Fv fragment, a diabody, a ScFv, a small modular immunopharmaceutical (SMIP), an affibody, an avimer, a nanobody, a domain antibody and/or single chains.

The antibody of the invention may be preferably of the IgG1, IgG2, IgG3, IgG4, IgM, IgA1, IgA2, IgAsec, IgD, and IgE antibody-type. It will be appreciated that antibodies that are generated need not initially possess such an isotype but, rather the antibody as generated can possess any isotype and that the antibody can be isotype-switched.

Preferably, the antibodies or fragments thereof according to the present invention are humanized antibodies.

A further embodiment of the present invention is the use of the aforesaid combination as medicament. Preferably in the treatment of neoplastic diseases.

In a preferred embodiment the neoplastic diseases are selected from pancreatic cancer, melanoma, bladder cancer, small cell lung cancer, head and neck cancer, breast cancer, prostate cancer and colon cancer, preferably pancreatic cancer.

A further embodiment is a pharmaceutical formulation comprising the combination of the present invention, optionally with at least one pharmaceutically acceptable excipient or carrier.

A further embodiment of the present invention is the use of said pharmaceutical formulation as a medicament.

A preferred embodiment of the present invention is the use of said pharmaceutical formulation in the treatment of neoplastic diseases, selected from pancreatic cancer, melanoma, bladder cancer, small cell lung cancer, head and neck cancer, breast cancer, prostate cancer and colon cancer.

Preferably, said neoplastic disease is pancreatic cancer.

The formulation of the present invention can be formulated in a form suitable for oral administration or in a form suitable for parenteral or topical administration.

In a preferred embodiment of the present invention, said oral form can be chosen from the following: tablets, capsules, solutions, suspensions, granules and oily capsules.

In a further preferred embodiment of the present invention, said topical form can be chosen from the following: cream, ointment, solution, suspension, pessary, nebuliser solution, spray, powder, or gel.

In a further preferred embodiment of this invention, said parenteral form can be either an aqueous buffer solution or an oily suspension.

Said parenteral administration include administration by intramuscular, intravenous, intradermal, subcutaneous, intraperitoneal, intranodal, or intrasplenic means.

In a further embodiment, the active principles of the combination of the present invention, that are the anti-BAG3 antibodies or a fragment thereof and the at least an inhibitor of the immune check point, can be administered simultaneously, separately or sequentially, also following different route of administration for each active principle.

According to a further embodiment of the present invention, the active principles of the combination can be administered together, through the same route of administration or through different route of administration, or they can be administered separately through the same route of administration or through different route of administration.

In a preferred aspect of the present invention, the anti-BAG3 antibody or a fragment thereof is formulated in oral form, preferably in form of tablets, capsules, solutions, suspensions, granules and oily capsules, while the at least an inhibitor of the immune check point is formulated parenterally, preferably an aqueous buffer solution or an oily suspension.

According to a preferred embodiment of the present invention, the formulation containing the anti-BAG3 antibodies or a fragment thereof are administered daily, preferably one or more time a day, while the formulation containing the at least one inhibitor of the immune check point are administered through parenteral route, preferably from one to several times a week.

The following examples are included to increase the understanding of the invention, without having any limiting effect of the invention.

### EXAMPLES

### Example 1—Chimerization and Humanization of AC2 Antibody

AC-2 murine antibody is produced by a hybridoma isolated from the hybridoma mother clone no. PD02009 deposited on the Dec. 17, 2002 at the Centro Biotecnologie Avanzate di Genova and disclosed in WO03/055908. Total RNA was extracted and RT-PCR performed to clone and sequence the variable regions of the antibody using conventional procedures (e.g., by using oligonucleotide probes that are capable of binding specifically to genes encoding the heavy and light chains of murine antibodies).

Based on sequence information of the variable region, heavy chain and light chain of AC-2 murine antibody (SEQ ID No. 1 and SEQ ID N. 2 for the amino acid sequences and SEQ ID 9 and SEQ ID N. 10 for the nucleotide sequences), different humanized variants of said region have been obtained by gene synthesis using standard procedures.

Sequences coding for the antibody variants were cloned in Evi-5 expression vector (Evitria AG, Switzerland) and expressed in CHO-K1 cells. For antibody chimerization, the murine constant regions were replaced with the human constant regions. One chimeric versions of the heavy chain (HC) was made in an IgG1 context.

For antibody humanization, Complementarity Determining Regions (CDRs) from the murine were grafted into a human antibody framework.

Twenty four humanized versions of the heavy chain (HC) were made in an IgG1 and LC-kappa context. Each version is characterized by specific point mutations in the FR.

### Example 2—Anti-BAG3 mAb Induces PD-1 Expression in Pancreatic Cancer Allografts in Immunocompetent Mice

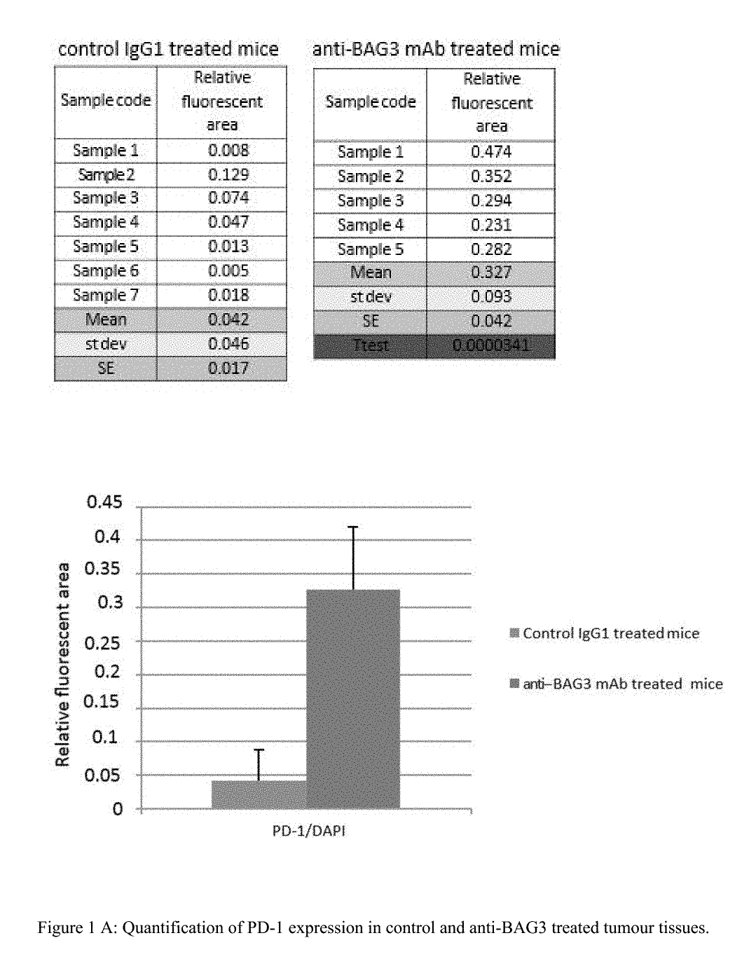

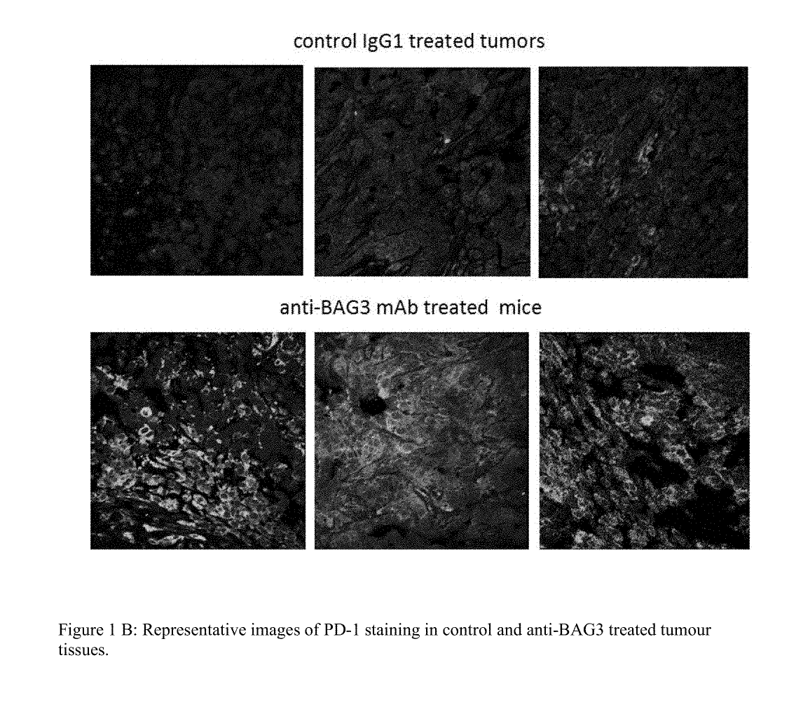

Tumours treated with anti-BAG3 antibody showed a significant higher expression of PD-1 protein (FIG. 1A). In FIG. 1B representative images are showed.

### Example 3—Anti-BAG3 mAb Induces PDL-1 Expression in Pancreatic Cancer Allografts in Immunocompetent Mice

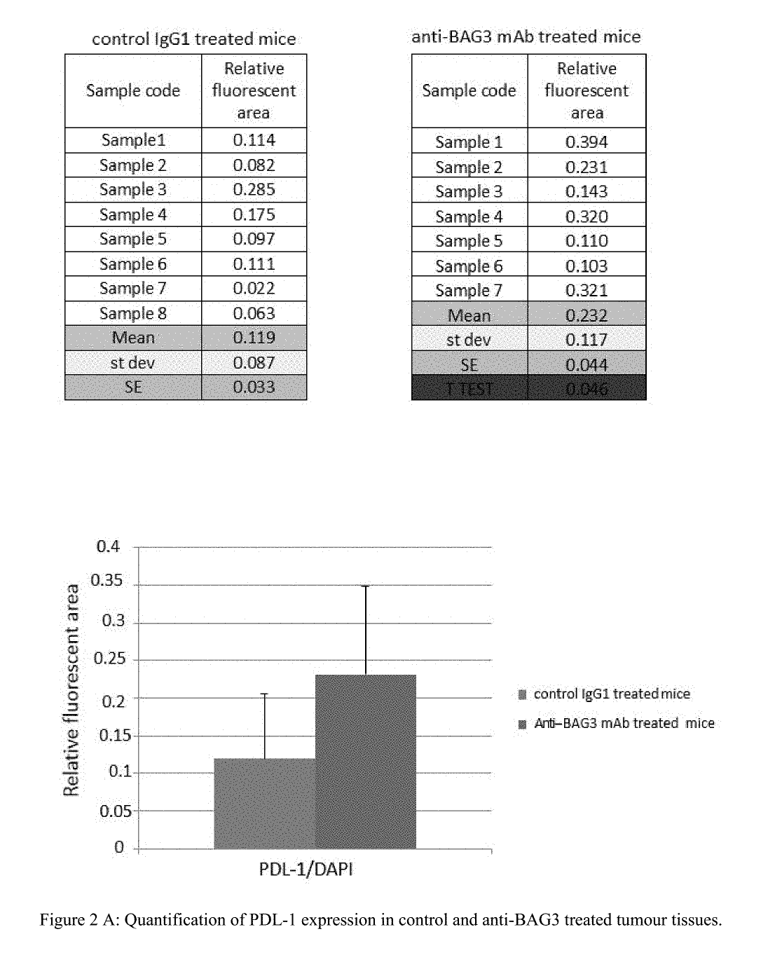

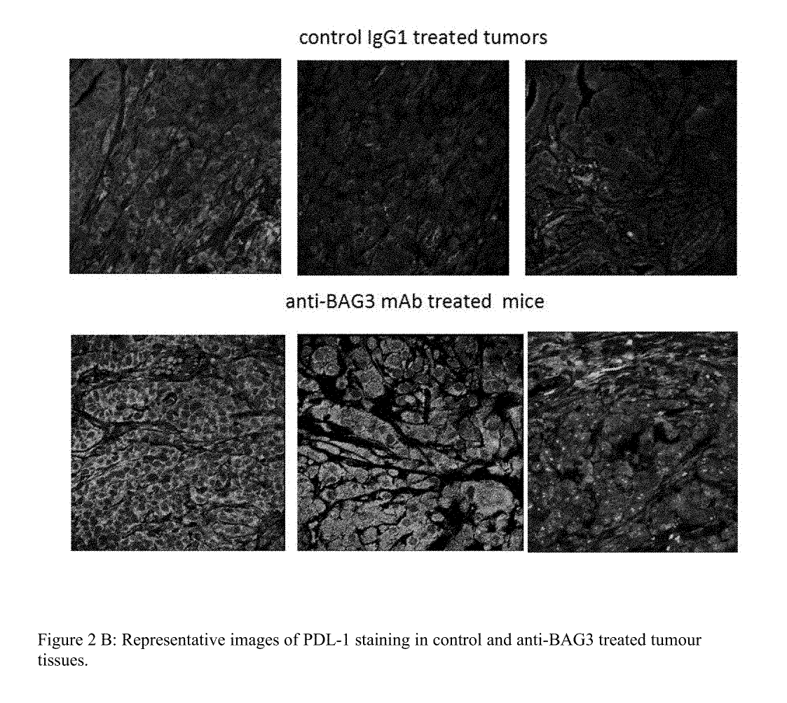

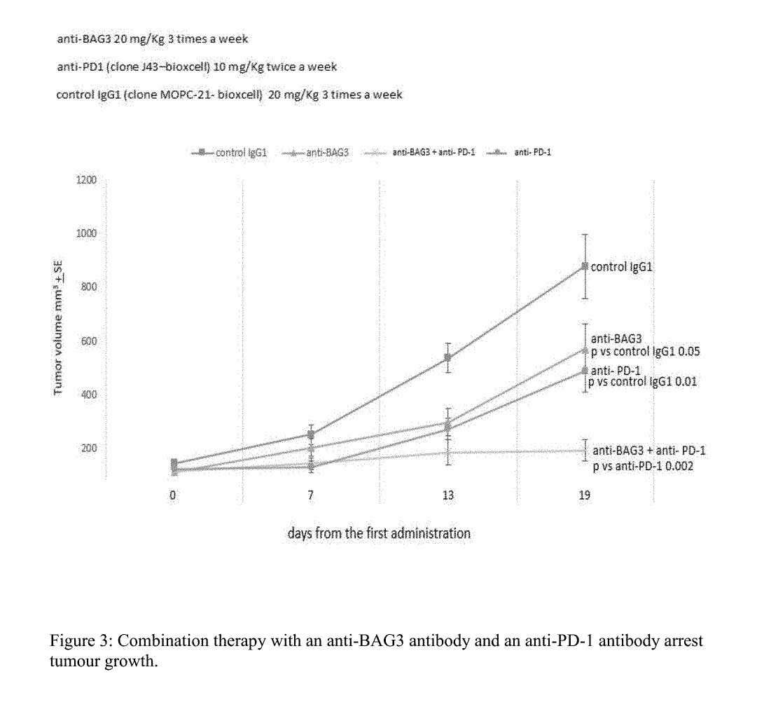

Tumours treated with anti-BAG3 antibody showed a significant higher expression of PDL-1 protein (FIG. 2A). In FIG. 2B representative images are showed.

### Example 4—Combination of Anti-BAG3 and Anti-PD1 Arrest Tumour Growth in Pancreatic Cancer Allografts in Immunocompetent Mice

In a syngeneic murine model of pancreatic cancer we showed that the combined treatment with an anti-BAG3 mAb and an anti-PD-1 mAb are able to arrest tumour growth.

Materials and Methods (Referred to Examples 2-4)

In Vivo Model:

mt4-2D murine cells (0.20×10⁶) were suspended in a solution 1:1 PBS 1×/matrigel and injected into the right and left flank of female C57BL6 mice (6 weeks old; Harlan Laboratories, Italy). After 10 days mice were divided in four arms consisting of 10 mice each in which tumour volume average was approximately 100 mm³. One group received i.p. injection of 20 mg Kg⁻¹ anti-BAG3 mAb in PBS 3 times a week alone or in combination with anti-PD-1 antibody (10 mg Kg⁻¹ twice a week, Bioxcell Clone: J-43), whereas the other received unrelated IgGs (Bioxcell Clone: MOPC-21) for 3 weeks. Another group received the administration of anti-PD-1 antibody. Animals were weighted and tumour volume measured by caliper twice weekly. At the end of the experiment all animals were sacrified and tumour samples collected for subsequent analysis.

Immunofluorescence:

At the end of the experiment tumours were paraffin embedded and sections analysed by immunofluorescence using an anti-PD-1 antibody or anti-PDL-1 (Abcam Cambridge, UK at 1:100). Immunofluorescence protocol included deparaffination in Clear-Rite™ 3 (ThermoScientific, Waltham, Mass. USA) rehydration through descending degrees of alcohol up to water, non-enzymatic antigen retrieval in in sodium citrate buffer 10 mM, 0.05% Tween, pH 6.0, for 3 minutes in microwave at 700 Watt. After washing, non-specific binding was blocked with 10% FBS in PBS 1×. Sections were then incubated with primary antibody anti-PD1 or anti-PDL-1 overnight at 4° C. in a humidified chamber. After another washing step, sections were incubated with the secondary antibody. Nuclei were counterstained with 1 μg ml-1 DAPI (Molecular Probes, Oregon, USA). Negative controls were performed using all reagents except the primary antibody. Images were acquired in sequential scan mode by using the same acquisitions parameters (laser intensities, gain photomultipliers, pinhole aperture, objective ×63, zoom 1) when comparing experimental and control material. For figure preparation, brightness and contrast of images were adjusted by taking care to leave a light cellular fluorescence background for visual appreciation of the lowest fluorescence intensity features and to help comparison among the different experimental groups. PD-1-positive or PDL-1-positive cells was calculated as ratio to DAPI staining using ImageJ software from at least ten images from ×63 field magnification

1. A composition comprising an anti-BAG3 antibody or a fragment thereof and at least one inhibitor of the immune check point.

2. The composition according to claim 1, characterized in that the least one inhibitor of the immune check point is an antibody, a protein, a small molecules and/or a si-RNA.

3. The composition according to claim 1, characterized in that the anti-BAG3 antibody or a fragment thereof is a humanized antibody that comprises:
a) a heavy chain amino acid sequence as encoded by SEQ ID NO: 12 or at least the variable domain thereof or an amino acid sequence having a sequence identity of at least 80% thereof, and
b) a light chain amino acid sequence as encoded by SEQ ID NO: 20 or at least the variable domain thereof or an amino acid sequence having a sequence identity of at least 80% thereof.

4. The composition according to claim 1, characterized in that the inhibitor of the immune check point is an antibody or a fragment thereof that binds to PD-1, PDL-1, A2AR, B7-H3, B7-H4, BTLA, CTLA-4, IDO, KIR, LAG3, TIM-3 or VISTA protein.

5. The composition according to claim 1, comprising one anti-BAG3 antibody or a fragment thereof and one anti-PD-1 antibody and/or one anti-PDL-1 antibody or a fragment thereof.

6. The composition according to claim 5, characterized in that said anti-PD-1 antibody is selected from Nivolumab and Pembrolizumab, and said anti-PDL-1 antibody is selected from Atezolizumab, Avelumab and Durvalumab.

7. The composition according to claim 1, characterized in that the anti-BAG3 antibody or the at least one inhibitor of the immune check point is a monoclonal antibody selected from the group consisting of a murine antibody, a recombinant antibody, humanized or fully human antibodies, chimeric antibody, and multispecific antibody, or a fragment thereof.

8. A pharmaceutical formulation comprising the composition according to claim 1, and optionally at least one pharmaceutically acceptable excipient or carrier.

9. (canceled)

10. A method for treating a neoplastic disease selected from pancreatic cancer, melanoma, bladder cancer, small cell lung cancer, head and neck cancer, breast cancer, prostate cancer and colon cancer, comprising the step of administering the composition of claim 1 to a person in need thereof.

11. The method according to claim 10, characterized in that the anti-BAG3 antibody or a fragment thereof and the at least one inhibitor of the immune check point are administered simultaneously, separately or sequentially.

12. The method according to claim 10, wherein the neoplastic disease is pancreatic cancer.

13. The composition according to claim 6, wherein said anti-PD-1 antibody is Nivolumab and said anti-PDL-1 antibody is selected from Atezolizumab.


SEQUENCE LISTING (26 sequences)

SEQ ID NO: 1 - protein, 115 aa, Mus musculus
EVQLQQSGAELVKPGASVKLSCTASGFNIKDTYMYWVKQRPEQGLEWIGGVDPANGNTRYDPKFQGKATLTADTSSSTAYLQLSSLTSEDTAVYYCGRDGAMDYWGQGTSVTVSS

SEQ ID NO: 2 - protein, 113 aa, Mus musculus
DIVMSQSPSSLAVSVGEKVTMSCKSSQSLLYSSNQKNYLAWHQQKPGQSPKLLIYWASTRESGVPDRFTGSGSGTDFTLTISSVKAEDLAIYYCQQYYTYPLTFGAGTKLELK

SEQ ID NO: 3 - protein, 10 aa, Mus musculus
GFNIKDTYMY

SEQ ID NO: 4 - protein, 17 aa, Mus musculus
GVDPANGNTRYDPKFQG

SEQ ID NO: 5 - protein, 6 aa, Mus musculus
DGAMDY

SEQ ID NO: 6 - protein, 17 aa, Mus musculus
KSSQSLLYSSNQKNYLA

SEQ ID NO: 7 - protein, 7 aa, Mus musculus
WASTRES

SEQ ID NO: 8 - protein, 9 aa, Mus musculus
QQYYTYPLT

SEQ ID NO: 9 - DNA, 345 bp, Mus musculus
  source 1..345: /mol_type=“unassigned DNA” /organism=“mouse”
gaggtccagctgcagcagagcggtgccgaactggtgaagccaggagcatccgtcaaactgtcttgtacagcatccgggtttaacattaaggacacctacatgtattgggtgaaacagaggccagagcagggcctggaatggatcggcggagtggaccccgctaacgggaatacacgatacgatcctaagttccagggaaaagccaccctgacagctgacacttccagctctaccgcatatctgcaactgagttccctgacatctgaggatactgccgtgtactattgcgggagggatggggctatggactactggggtcaggggacttccgtcactgtctcgagc

SEQ ID NO: 10 - DNA, 339 bp, Mus musculus
  source 1..339: /mol_type=“unassigned DNA” /organism=“mouse”
gatattgtgatgtctcagtccccaagcagcctggcagtctcagtcggcgaaaaggtgaccatgtcctgtaaatcctctcagtccctgctgtactccagcaaccagaagaattatctggcatggcaccagcagaagcccggacagagtcctaaactgctgatctactgggccagcacaagggagtctggcgtgccagaccggttcactggctcaggctccgggaccgattttaccctgacaatctctagtgtcaaagccgaagacctggctatctactattgccagcagtattacacttatcctctgacatttggagcagggactaaactggaactgaag

SEQ ID NO: 11 - DNA, 1416 bp, Mus musculus
  source 1..1416: /mol_type=“unassigned DNA” /organism=“mouse”
gcggccgccatgaattttggactgaggctgattttcctggtgctgaccctgaaaggcgtccagtgtcaggtgcagctggtccagagcggggcagaggtgaagaaaccaggtgccagcgtgaaggtctcttgcaaagccagtggcttcaacatcaaggacacatacatgtattgggtgcgacaggcccctggccagggtctggaatggatgggcggcgtggaccccgcaaatggaaatactagatacgatcctaaatttcagggaagggtgaccatgacacgggacacttcaacctcgacggtctatatggagctgtccagcctgagatccgaagatacagccgtgtactattgtgcccgcgacggggctatggattactggggccagggaactctggtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 12 - protein, 445 aa, Mus musculus
QVQLVQSGAEVKKPGASVKVSCKASGFNIKDTYMYWVRQAPGQGLEWMGGVDPANGNTRYDPKFQGRVTMTRDTSTSTVYMELSSLRSEDTAVYYCARDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1416 bp, Homo sapiens
  source 1..1416: /mol_type=“unassigned DNA” /organism=“Homo sapiens”
gcggccgccatgaattttggactgaggctgattttcctggtgctgaccctgaaaggcgtccagtgtcaggtgcagctggtccagtctggagctgaggtgaagaaaccaggagcctccgtgaaggtctcttgcaaagccagtggcttcaacatcaaggacacatacatgtattgggtgcgacaggcccctggccagggtctggaatggatgggcggcgtggaccccgcaaatggaaatactagatacgatcctaaatttcaaggcagggtgaccctgacacgggacacttcaacctcgacggtctatatggagctgtccagcctgagatccgaagatacagcagtgtactattgtgggcgcgacggtgctatggactactggggccagggaactctggtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 14 - protein, 445 aa, Homo sapiens
QVQLVQSGAEVKKPGASVKVSCKASGFNIKDTYMYWVRQAPGQGLEWMGGVDPANGNTRYDPKFQGRVTLTRDTSTSTVYMELSSLRSEDTAVYYCGRDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1416 bp, Homo sapiens
  source 1..1416: /mol_type=“unassigned DNA” /organism=“Homo sapiens”
gcggccgccatgaattttggactgaggctgattttcctggtgctgaccctgaaaggcgtccagtgtcaggtgcagctggtccagtctggagctgaggtgaagaaaccaggagcctccgtgaaggtctcttgcacagctagtggcttcaacatcaaggacacttacatgtattgggtgaaacaggcccctggccagggtctggaatggattggcggcgtggaccccgcaaacgggaataccagatacgatcctaagtttcaaggcaaagccaccctgacaagggacacttcaacctcgacggtgtatatggagctgtccagcctgaggtccgaagatacagcagtgtactattgtgggcgggacggtgctatggactactggggccagggaactctggtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 16 - protein, 445 aa, Homo sapiens
QVQLVQSGAEVKKPGASVKVSCTASGFNIKDTYMYWVKQAPGQGLEWIGGVDPANGNTRYDPKFQGKATLTRDTSTSTVYMELSSLRSEDTAVYYCGRDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1416 bp, Homo sapiens
  source 1..1416: /mol_type=“unassigned DNA” /organism=“Homo sapiens”
gcggccgccatgaattttggactgaggctgattttcctggtgctgaccctgaaaggcgtccagtgtgaggtgcagctggtccagagtggggcagaagtgaagaaaccaggtgccacagtgaagatctcatgcaaagtctccggcttcaacattaaggacacttacatgtattgggtgcagcaggcccccggcaagggtctggagtggatgggcggcgtggaccccgctaacggcaataccagatacgatcctaagtttcaaggacgggtgaccatcacagctgacactagcaccgatacggcatatatggagctgtccagcctgagatctgaagatacagcagtgtactattgtgccagggacggggctatggattactggggccagggaactctggtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatgatagtaaaagctt

SEQ ID NO: 18 - protein, 445 aa, Homo sapiens
EVQLVQSGAEVKKPGATVKISCKVSGFNIKDTYMYWVQQAPGKGLEWMGGVDPANGNTRYDPKFQGRVTITADTSTDTAYMELSSLRSEDTAVYYCARDGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 741 bp, Homo sapiens
  source 1..741: /mol_type=“other DNA” /organism=“Homo sapiens”
gcggccgccatgaattttggactgaggctgattttcctggtgctgaccctgaaaggcgtccagtgtgacatcgtgatgacacagtcacctgattccctggcagtcagtctgggcgagagagccactattaactgcaagtccagccagtctctgctgtactctagtaaccagaaaaattacctggcttggtatcagcagaagccagggcagccccctaaactgctgatctattgggcaagcaccagggaatctggagtgcccgaccggttcagcggttctggcagtggaacagattttaccctgacaatttcatccctgcaagccgaggacgtggctgtctactattgtcagcagtactatacttacccactgaccttcggcggagggaccaagctcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 20 - protein, 220 aa, Homo sapiens
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 741 bp, Homo sapiens
  source 1..741: /mol_type=“unassigned DNA” /organism=“Homo sapiens”
gcggccgccatgaattttggactgaggctgattttcctggtgctgaccctgaaaggcgtccagtgtgacatcgtgatgacacagtcacctgattccctggcagtcagtctgggcgagagagccactattaactgcaagtccagccagtctctgctgtactctagtaaccagaaaaattatctggcttggcaccagcagaagccagggcagccccctaaactgctgatctactgggcaagcaccagggaatctggagtgcccgaccggttcagcggttctggcagtggaacagattttaccctgacaatttcatccctgcaagccgaggacgtggctgtctactattgtcagcagtactatacttatccactgaccttcggcggagggaccaagctcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 22 - protein, 220 aa, Homo sapiens
DIVMTQSPDSLAVSLGERATINCKSSQSLLYSSNQKNYLAWHQQKPGQPPKLLIYWASTRESGVPDRFSGSGSGTDFTLTISSLQAEDVAVYYCQQYYTYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 741 bp, Homo sapiens
  source 1..741: /mol_type=“unassigned DNA” /organism=“Homo sapiens”
gcggccgccatgaattttggactgaggctgattttcctggtgctgaccctgaaaggcgtccagtgtgacatcgtgatgacacagtcacctgattccctggcagtctccctgggcgagagagccactatgagttgcaagtccagccagtctctgctgtactctagtaaccagaaaaattatctggcttggcaccagcagaagccagggcagccccctaaactgctgatctactgggcaagcaccagggaatctggagtgcccgaccggttcagcggttctggcagtggaacagattttaccctgacaatttcatccctgcaagccgaggacgtggctgtctactattgtcagcagtactatacttatccactgaccttcggcggagggaccaagctcgagatcaagcgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 24 - protein, 220 aa, Homo sapiens
DIVMTQSPDSLAVSLGERATMSCKSSQSLLYSSNQKNYLAWHQQKPGQPPKLLIYWASTRESGVPDRFSGSGSGTDFTLTISSLQAEDVAVYYCQQYYTYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 741 bp, Homo sapiens
  source 1..741: /mol_type=“unassigned DNA” /organism=“Homo sapiens”
gcggccgccatgaattttggactgaggctgattttcctggtgctgaccctgaaaggcgtccagtgtgacatcgtgatgacacagtcacctgattccctggcagtctccctgggcgagagagccactatgagttgcaagtccagccagtctctgctgtactctagtaaccagaaaaattatctggcttggcaccagcagaagccaggacagccccctaaactgctgatctactgggcaagcaccagggaatctggcgtgcccgaccggttcagcggctctggaagtgggacagattttaccctgacaatctcatccctgcaagccgaggacctggctatctactattgtcagcagtactatacttatccactgaccttcggtgccggcaccaagctcgagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctgatagtaaaagctt

SEQ ID NO: 26 - protein, 220 aa, Homo sapiens
DIVMTQSPDSLAVSLGERATMSCKSSQSLLYSSNQKNYLAWHQQKPGQPPKLLIYWASTRESGVPDRFSGSGSGTDFTLTISSLQAEDLAIYYCQQYYTYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC